(19) United States
(12) Patent Application Publication
(10) Pub. No.: US 2010/0196461 A1
(43) Pub. Date: Aug. 5, 2010

(54) RESUSCITATION FLUID

(76) Inventor: Cuthbert O. Simpkins, Shreveport, LA (US)

Correspondence Address: ANDREWS KURTH LLP, 1350 I STREET, N.W., SUITE 1100, WASHINGTON, DC, 20005, US

(21) Appl. No.: 12/696,107

(22) Filed: Jan. 29, 2010

Related U.S. Application Data
(60) Provisional application No. 61/202,124, filed on Jan. 30, 2009.

(51) Int. Cl. A61K 9/127; A61K 33/00; A61K 35/14; A61K 35/16; A61P 7/08; A01N 1/02; A61J 1/05; A61M 37/00; A61M 25/00

(52) U.S. Cl. 424/450; 424/613; 424/529; 424/530; 435/1.2; 604/403; 604/24; 604/523

(57) ABSTRACT

A method for treating conditions related to lack of blood supply with a resuscitation fluid is disclosed. The resuscitation fluid contains a lipophilic component and a polar liquid carrier. The lipophilic component forms an emulsion with the polar liquid carrier. The resuscitation fluid can be used to increase the blood pressure and to carry oxygen and other lipophilic gases to tissues. The resuscitation fluid can also be used for preserving the biological integrity of donor organs for transplantation.

### CROSS-REFERENCE TO RELATED APPLICATIONS

This application claims priority of U.S. Provisional Application Ser. No. 61/202,124 filed Jan. 30, 2009, which is incorporated by reference in its entirety.

### TECHNICAL FIELD

The technical field is medical treatment and, in particular, methods and compositions for treating conditions related to lack of blood supply.

### BACKGROUND

When a large amount of blood is lost, it is critical to immediately replace the lost volume with a volume expander to maintain circulatory volume, so that the remaining red blood cells can still oxygenate body tissue. In extreme cases, an infusion of real blood or blood substitute may be needed to maintain adequate tissue oxygenation in the affected individual. A blood substitute differs from a simple volume expander in that the blood substitute has the ability to carry oxygen like real blood.

Currently employed blood substitutes use either perfluorocarbons (PFCs) or hemoglobins as the oxygen carrier. PFCs are compounds derived from hydrocarbons by replacing the hydrogen atoms in the hydrocarbons with fluorine atoms. PFCs are capable of dissolving relatively high concentrations of oxygen. However, medical applications require high purity perfluorocarbons. Impurities with nitrogen bonds can be highly toxic. Hydrogen-containing compounds (which can release hydrogen fluoride) and unsaturated compounds must also be excluded. The purification process is complex and costly.

Hemoglobin is the iron-containing oxygen-transport metalloprotein in the red blood cells. Pure hemoglobin separated from red blood cells, however, cannot be used since it causes renal toxicity. Various modifications, such as cross-linking, polymerization, and encapsulation, are needed to convert hemoglobin into a useful and safe artificial oxygen carrier. The resulting products, often referred to as HBOCs (Hemoglobin Based Oxygen Carriers), are expensive and are directly toxic to cells.

Gases other than oxygen are important in the regulation of vascular function and cellular metabolism. Examples are nitric oxide and carbon monoxide. Nitirc oxide has been shown to play a significant role in the maintaining the patency of the microcirculation. Carbon monoxide has been shown to have an antiapoptotic effect. Therapeutic quantities of these and other gases should also be provided by the ideal resuscitation fluid.

Therefore, there still exists a need for a lower-cost resuscitation fluid that functions as a volume expander but is also capable of carrying a large amount of oxygen and, optionally, therapeutic amounts of other gases.

### SUMMARY

A method for treating conditions related to lack of blood supply is disclosed. The method includes administering to a subject in need of such treatment an effective amount of a resuscitation fluid that contains a lipophilic component and polar liquid carrier. The lipophilic component forms an emulsion with the polar liquid carrier.

Also disclosed is a method for preserving the biological integrity of an organ of a mammalian donor organism. The method includes perfusing the organ with an effective amount of a resuscitation fluid containing a lipophilic component and a polar liquid carrier, wherein the lipophilic component forms an emulsion polar liquid carrier.

Also disclosed is a resuscitation fluid. The resuscitation fluid contains an oxygenated lipophilic component emulsion and a buffering agent. Other hydrophobic gases, such as nitric oxide, carbon monoxide and xenon, may also be loaded onto the lipophilic component of the resuscitation fluid.

Also disclosed is a resuscitation kit. The resuscitation kit contains a lipid based resuscitation fluid having a lipophilic component and a polar liquid carrier and a device for loading oxygen and/or other gases onto the lipophilic component.

### DESCRIPTION OF THE DRAWINGS

The detailed description will refer to the following drawings, wherein like numerals refer to like elements, and wherein:

FIG. 1 is a diagram showing systolic blood pressure in mice treated with different resuscitation fluids after severe hemorrhagic shock. The mean blood pressure immediately before infusion across all experiments of resuscitation fluid was 4.6+/−1.2. The systolic pressure immediately before infusion of the fluid is subtracted out.

FIG. 2 is a diagram showing diastolic blood pressure in mice treated with different resuscitation fluids after severe hemorrhagic shock. The diastolic pressure immediately before infusion of the fluid is subtracted out.

FIG. 3 is a diagram showing systolic blood pressure in mice treated with a resuscitation fluid of different volumes after severe hemorrhagic shock. The systolic pressure immediately before infusion of the fluid is subtracted out.

FIG. 4 is a diagram showing diastolic blood pressure in mice treated with a resuscitation fluid of different volumes after severe hemorrhagic shock. The diastolic pressure immediately before infusion of the fluid is subtracted out.

FIG. 5 is a diagram showing systolic blood pressure in mice treated with albumin-containing resuscitation fluids and mice treated with shed blood after severe hemorrhagic shock. Data is shown as the percentage of mean pre-hemorrhage blood pressure.

### DETAILED DESCRIPTION

One aspect of the present invention relates to a resuscitation fluid composition for treating conditions related to lack of blood supply with a resuscitation fluid. The resuscitation fluid comprises a lipophilic component and a polar liquid carrier. The lipophilic component is dispersed in the polar liquid carrier to form an emulsion that typically contains micelles or liposomes with a polar outer surface and an inner hydrophobic space. The resuscitation fluid can be used to increase blood pressure and to carry oxygen to tissues in the absence of natural or modified hemoglobin.

In another embodiment, the resuscitation fluid comprises a lipophilic component encapsulated by erythrocyte ghost.

The conditions related to lack of blood supply include, but are not limited to, hypovolemia caused by bleeding, dehydration, vomiting, severe burns, systemic inflammatory response syndrome (SIRS) and drugs such as diuretics or vasodilators. Severe hypovolemia may occur in conjunction with capillary leak (CL), which is present in different conditions such as multiorgan dysfunction (MODS), sepsis, trauma, burn, hemorrhagic shock, post-cardiopulmonary bypass, pancreatitis and systemic capillary leak syndrome, and causes morbidity and mortality among a large number of hospital patients.

### Lipophilic Component

The oxygen-carrying lipophilic component can be any pharmaceutically acceptable lipophilic or pharmaceutically acceptable amphiphilic material that is capable of forming an emulsion with a polar liquid, including but not limited to, lipids and amphiphiles. The lipid and/or amphiphile molecules may aggregate as micelles, liposomes, or micelles/liposomes carrying the same or another lipophilic substance in the hydrophobic core. The oxygen or other gases may be carried in the membrane of the micelles/liposomes, in the hydrophobic core of the micelles/liposomes, and in lipophilic substance encapsulated by the micelle/liposomes. Lipophilic gases other than oxygen such as nitric oxide, carbon monoxide, hydrogen sulfide or xenon may also be carried in a similar manner. As used herein, the term “lipid” refers to a fat-soluble material that is naturally occurring, or non-naturally occurring. Examples of lipids include but are not limited to, fatty acyls, glycerolipids, phospholipids, sphingolipids, sterol lipids, prenol lipids, saccharolipids, polyketides, non-natural lipid(s), cationic lipid(s), amphipathic alkyl amino acid derivative, adialkyldimethylammonium, polyglycerol alkyl ethers, polyoxyethylene alkyl ethers, and mixtures thereof. In certain embodiments, the lipophilic component comprises soybean oil. In one embodiment, the lipophilic component is a mixture of soybean oil and egg yolk phospholipids, such as those used in Intralipid® (marketed and sold by Baxter International Inc., Deerfield, Ill.).

Examples of the glycolipids include glyceroglycolipids and sphingoglycolipids. Examples of glyceroglycolipids include digalactosyl diglycerides (such as digalactosyl dilauroyl glyceride, digalactosyl dimyristoyl glyceride, digalactosyl dipalmitoyl glyceride, and digalactosyl distearoyl glyceride) and galactosyl diglycerides (such as galactosyl dilauroyl glyceride, galactosyl dimyristoyl glyceride, galactosyl dipalmitoyl glyceride, and galactosyl distearoyl glyceride). Examples of sphingoglycolipids include galactosyl cerebroside, lactosyl cerebroside, and ganglioside.

Examples of phospholipids include natural or synthetic phospholipids such as phosphatidylcholine, phosphatidylethanolamine, phosphatidylserine, phosphatidic acid, phosphatidylglycerol, phosphatidylinositol, lisophosphatidylcholine, sphingomyelin, egg yolk lecithin, soybean lecithin, and a hydrogenated phospholipid.

Examples of the sterols include cholesterol, cholesterol hemisuccinate, 3β-[N—(N′, N′-dimethylaminoethane)carbamoyl]cholesterol, ergosterol, and lanosterol.

As used herein, the term “amphiphiles” refers to a chemical compound possessing both hydrophilic and lipophilic properties. Examples of amphiphiles include, but are not limited to, naturally-occurring amphiphiles such as phospholipids, cholesterol, glycolipids, fatty acids, bile acids, and saponins; and synthetic amphiphiles.

In certain embodiments, the lipid component comprises an unsaturated fatty acid with one or more alkenyl functional groups in cis or trans configuration. A cis configuration means that adjacent hydrogen atoms or other groups are on the same side of the double bond. In a trans configuration theses moieties are on different sides of the double bond. The rigidity of the double bond freezes its conformation and, in the case of the cis isomer, causes the chain to bend and restricts the conformational freedom of the fatty acid. In general, the more double bonds the chain has, the less flexibility it has. When a chain has many cis bonds, it becomes quite curved in its most accessible conformations. For example, oleic acid, with one double bond, has a “kink” in it, while linoleic acid, with two double bonds, has a more pronounced bend. Alpha-linolenic acid, with three double bonds, favors a hooked shape. The effect of this is that in restricted environments, such as when fatty acids are part of a phospholipid in a lipid bilayer, or triglycerides in lipid droplets, cis bonds limit the ability of fatty acids to be closely packed and therefore could affect the melting temperature of the membrane or of the fat. In some embodiments, the lipid component comprises up to 1%, 2%, 5%, 10%, 15%, 20%, 25%, 30%, 35%, 40%, 45%, 50%, 55%, 60%, 65%, 70%, 75%, 80%, 85%, 90%, 95% or 99% (w/w) unsaturated fatty acid(s) that have one or more alkenyl functional groups in cis configuration.

Examples of cis-unsaturated fatty acids include, but are not limited to, obtusilic acid, linderic acid, tsuzuic acid, palmito-oleic acid, oleic acid, elaidic acid, vaccenic acid, petroselinic acid, gadoleic acid, eicosenoic acid, erucic acid, cetoleic acid, nervonic acid, ximenic acid and lumepueic acid; n-3 type unsaturated fatty acids such as α-linolenic acid, stearidonic acid, eicosatetraenoic acid, eicosapentaenoic acid, docosapentaenoic acid and docosahexaenoic acid; n-6 type unsaturated fatty acids such as linoleic acid, linoelaidic acid, γ-linolenic acid, bis-homo-γ-linolenic acid and arachidonic acid; conjugated fatty acids such as conjugated linoleic acid and α-eleostearic acid; fatty acids carrying double bonds at the 5-position thereof such as pinolenic acid, sciadonic acid, juniperic acid and columbinic acid; polyvalent unsaturated fatty acids, other than those listed above, such as hiragonic acid, moroctic acid, clupanodonic acid and nishinic acid; branched fatty acids such as isobutyric acid, isovaleric acid, iso acid and anti-iso acid; hydroxy fatty acids such as α-hydroxy acid, β-hydroxy acid, mycolic acid and polyhydroxy acid; epoxy-fatty acids; keto-fatty acids; and cyclic fatty acids.

### Polar Liquid Carrier

The polar liquid carrier can be any pharmaceutically acceptable polar liquid that is capable of forming an emulsion with the lipid. The term “pharmaceutically acceptable” refers to molecular entities and compositions that are of sufficient purity and quality for use in the formulation of a composition or medicament of the present invention and that, when appropriately administered to an animal or a human, do not produce an adverse, allergic or other untoward reaction. Since both human use (clinical and over-the-counter) and veterinary use are equally included within the scope of the present invention, a pharmaceutically acceptable formulation would include a composition or medicament for either human or veterinary use. In one embodiment, the polar liquid carrier is water or a water based solution. In another embodiment, the polar liquid carrier is a non-aqueous polar liquid such as dimethyl sulfoxide, polyethylene glycol and polar silicone liquids.

A water-based solution generally comprises a physiologically compatible electrolyte vehicle isosmotic with whole blood. The carrier can be, for example, physiological saline, a saline-glucose mixture, Ringer's solution, lactated Ringer's solution, Locke-Ringer's solution, Krebs-Ringer's solution, Hartmann's balanced saline, heparinized sodium citrate-citric acid-dextrose solution, and polymeric plasma substitutes, such as polyethylene oxide, polyvinyl pyrrolidone, polyvinyl alcohol and ethylene oxide-propylene glycol condensates. The resuscitation fluid may additionally comprise other constituents such as pharmaceutically-acceptable carriers, diluents, fillers and salts, the selection of which depends on the dosage form utilized, the condition being treated, the particular purpose to be achieved according to the determination of the ordinarily skilled artisan in the field and the properties of such additives.

### Rigid Nonplanar Molecules

The resuscitation fluid may further comprise molecules with a rigid nonplanar structure. Such molecules will create greater irregularity and more space for gas molecules in the micelle structure, thereby modifying the gas carrying capacity of the micelles. Examples of such molecules include, but are not limited to, (+) naloxone, (+) morphine, and (+) naltrexone.

In one embodiment, molecules with a rigid nonplanar structure is (+) naloxone which, unlike the opiate receptor antagonist (−) naloxone, does not bind to opiate receptor and will not increase pain as (−) naloxone would. In another embodiment, (+) naloxone is used at a concentration of 10⁻⁵-10⁻⁴ M. In another embodiment, (+) naloxone is used at a concentration of 10⁻⁴ M or higher.

Upon resuscitation, an inflammatory process may be triggered in reperfused tissues (ischemic-reperfusion injury) causing endothelial cell (EC) injury and capillary leak (CL). In sepsis and other diseases, systemic inflammation may be triggered by the disease and in a similar sequence leads to EC injury, CL, and ultimately hypovolemic shock that requires resuscitation. Accordingly, in one embodiment, (+) naloxone is used at a concentration range that produces anti-inflammatory effect at 10⁻⁵-10⁻⁴ M.

Molecules with a nonplanar structure also include organic molecules with branched structures. Examples of such molecules include, but are not limited to, tri-n-octylamine, tri-n-hexylamine, boric acid, tris(3,5,-dimethyl-4-heptyl) ester, metal complexed and non-metal complexed deuteroporphyrin dimethyl esters and their derivatives, hexaphenylsilole, and silicone polymers.

### Plasma Component

The resuscitation fluid may further comprise a plasma component. In one embodiment, the plasma is an animal plasma. In another embodiment, the plasma is human plasma. Although not wishing to be bound by any particular scientific theory, it is believed that the administration of blood substitutes may dilute the concentration of coagulation factors to an undesirable level. Accordingly, using plasma as the diluent for the oxygen carrying component avoids this problem. Plasma can be collected by any means known in the art, provided that red cells, white cells and platelets are essentially removed. Preferably, it is obtained using an automated plasmaphoresis apparatus. Plasmaphoresis apparatuses are commercially available and include, for example, apparatuses that separate plasma from the blood by ultrafiltration or by centrifugation. An ultrafiltration-based plasmaphoresis apparatus such as manufactured by Auto C, A200 (Baxter International Inc., Deerfield, Ill.) is suitable because it effectively removes red cells, white cells and platelets while preserving coagulation factors.

Plasma may be collected with an anticoagulant, many of which are well known in the art. Preferred anti-coagulants are those that chelate calcium such as citrate. In one embodiment, sodium citrate is used as an anticoagulant at a final concentration of 0.2-0.5%, preferably 0.3-0.4%, and most preferably at 0.38%. The plasma may be fresh, frozen, pooled and/or sterilized. While plasma from exogenous sources may be preferred, it is also within the present invention to use autologous plasma that is collected from the subject prior to formulation and administration of the resuscitation fluid.

In addition to plasma from natural sources, synthetic plasma may also be used. The term “synthetic plasma,” as used herein, refers to any aqueous solution that is at least isotonic and that further comprises at least one plasma protein.

### Oncotic Agent

In one embodiment, the resuscitation fluid further contains an oncotic agent in addition to the lipid micelles. The oncotic agent is comprised of molecules whose size is sufficient to prevent their loss from circulation by traversing the fenestrations of the capillary bed into the interstitial spaces of the tissues of the body. Examples of oncotic agents include, but are not limited to, dextran (e.g., a low-molecular-weight dextran), dextran derivatives (e.g., carboxymethyl dextran, carboxydextran, cationic dextran, and dextran sulfate), hydroxyethyl starch, hydroxypropyl starch, branched, unsubstituted or substituted starch, gelatin (e.g., modified gelatin), albumin (e.g., human plasma, human serum albumin, heated human plasma protein, and recombinant human serum albumin), PEG, polyvinyl pyrrolidone, carboxymethylcellulose, acacia gum, glucose, a dextrose (e.g., glucose monohydrate), oligosaccharides (e.g., oligosaccharide), a polysaccharide degradation product, an amino acid, and a protein degradation product. Among those, particularly preferable are low-molecular-weight dextran, hydroxyethyl starch, modified gelatin, and recombinant albumin.

In one embodiment, the oncotic agent is about 5% (w/v) albumin. In another embodiment, the oncotic agent is a polysaccharide, such as Dextran, in a molecular weight range of 30,000 to 50,000 daltons (D). In yet another embodiment, the oncotic agent is a polysaccharide, such as Dextran, in a molecular weight range of 50,000 to 70,000 D. High molecular weight dextran solutions are more effective in preventing tissue swelling due to their lower rates of leakage from capillaries. In one embodiment, the concentration of the polysaccharide is sufficient to achieve (when taken together with chloride salts of sodium, calcium and magnesium, organic ion from the organic salt of sodium and hexose sugar discussed above) colloid osmotic pressure approximating that of normal human serum, about 28 mm Hg.

### Crystalloid Agent

The resuscitation fluid may also comprise a crystalloid agent. The crystalloid agent can be any crystalloid which, in the form of the resuscitation fluid composition, is preferably capable of achieving an osmolarity greater than 800 mOsm/l, i.e. it makes the resuscitation fluid “hypertonic”. Examples of suitable crystalloids and their concentrations in the resuscitation fluid include, but are not limited to, 3% w/v NaCl, 7% NaCl, 7.5% NaCl, and 7.5% NaCl in 6% w/v dextran. In one embodiment, the resuscitation fluid has an osmolarity of between 800 and 2400 mOsm/l.

When the resuscitation fluid further comprises a crystalloid and is hypertonic, the resuscitation fluid may provide improved functionality for rapid recovery of hemodynamic parameters over other blood substitute compositions, which include a colloid component. Small volume highly hypertonic crystalloid infusion (e.g., 1-10 ml/kg) provides significant benefits in the rapid and sustained recovery of acceptable hemodynamic parameters in controlled hemorrhage. In another embodiment, the lipid emulsion used is Intralipid®. In another embodiment, the lipid emulsion used is 20% Intralipid®. In one embodiment, the lipid comprises anti-inflammatory lipids such as omega-3 fatty acids.

### Anti-Inflammatory and Immunomodulatory Agent

In one embodiment, the resuscitation fluid of the present invention further includes an anti-inflammatory or immunomodulatory agent. Examples of the anti-inflammatory agent shown to inhibit reactive oxygen species including, but are not limited to, histidine, albumin, (+) naloxone, prostaglandin D₂, molecules of the phenylalkylamine class. Other anti-inflammatory compounds and immunomodulatory drug include interferon; interferon derivatives comprising betaseron, β-interferon; prostane derivatives comprising iloprost, cicaprost; glucocorticoids comprising cortisol, prednisolone, methyl-prednisolone, dexamethasone; immunsuppressives comprising cyclosporine A, methoxsalene, sulfasalazine, azathioprine, methotrexate; lipoxygenase inhibitors comprising zileutone, MK-886, WY-50295, SC-45662, SC-41661A, BI-L-357; leukotriene antagonists; peptide derivatives comprising ACTH and analogs thereof; soluble TNF-receptors; anti-TNF-antibodies; soluble receptors of interleukins or other cytokines; antibodies against receptors of interleukins or other cytokines, T-cell-proteins; and calcipotriols and analogues thereof taken either alone or in combination.

### Electrolytes

In one embodiment, the resuscitation fluid of the present invention includes one or more electrolytes. The electrolyte to be used in the present invention typically includes various electrolytes to be used for medicinal purposes. Examples of the electrolyte include sodium salts (e.g., sodium chloride, sodium hydrogen carbonate, sodium citrate, sodium lactate, sodium sulfate, sodium dihydrogen phosphate, disodium hydrogen phosphate, sodium acetate, sodium glycerophosphate, sodium carbonate, an amino acid sodium salt, sodium propionate, sodium β-hydroxybutyrate, and sodium gluconate), potassium salts (e.g., potassium chloride, potassium acetate, potassium gluconate, potassium hydrogen carbonate, potassium glycerophosphate, potassium sulfate, potassium lactate, potassium iodide, potassium dihydrogen phosphate, dipotassium hydrogen phosphate, potassium citrate, an amino acid potassium salt, potassium propionate, and potassium β-hydroxybutyrate), calcium salts (e.g., calcium chloride, calcium gluconate, calcium lactate, calcium glycerophosphate, calcium pantothenate, and calcium acetate), magnesium salts (e.g., magnesium chloride, magnesium sulfate, magnesium glycerophosphate, magnesium acetate, magnesium lactate, and an amino acid magnesium salt), ammonium salts (e.g., ammonium chloride), zinc salts (e.g., zinc sulfate, zinc chloride, zinc gluconate, zinc lactate, and zinc acetate), iron salts (e.g., iron sulfate, iron chloride, and iron gluconate), copper salts (e.g., copper sulfate), and manganese salts (for example, manganese sulfate). Among those, particularly preferable are sodium chloride, potassium chloride, magnesium chloride, disodium hydrogen phosphate, dipotassium hydrogen phosphate, potassium dihydrogen phosphate, sodium lactate, sodium acetate, sodium citrate, potassium acetate, potassium glycerophosphate, calcium gluconate, calcium chloride, magnesium sulfate, and zinc sulfate.

Concentration of calcium, sodium, magnesium and potassium ion is typically within the range of normal physiological concentrations of said ions in plasma. In general, the desired concentration of these ions is obtained from the dissolved chloride salts of calcium, sodium and magnesium. The sodium ions may also come from a dissolved organic salt of sodium that is also in solution.

In one embodiment, the sodium ion concentration is in a range from 70 mM to about 160 mM. In another embodiment, the sodium ion concentration is in a range of about 130 to 150 mM.

In one embodiment, the concentration of calcium ion is in a range of about 0.5 mM to 4.0 mM. In another embodiment, the concentration of calcium ion is in a range of about 2.0 mM to 2.5 mM.

In one embodiment, the concentration of magnesium ion is in a range of 0 to 10 mM. In another embodiment, the concentration of magnesium ion is in a range of about 0.3 mM to 0.45 mM. It is best not to include excessive amounts of magnesium ion in the resuscitation fluid of the invention because high magnesium ion concentrations negatively affect the strength of cardiac contractile activity. In a preferred embodiment of the invention, the solution contains subphysiological amounts of magnesium ion.

In one embodiment, the concentration of potassium ion is in a subphysiological range of between 0-5 mEq/l K⁺ (0-5 mM), preferably 2-3 mEq/l K⁺ (2-3 mM). Thus, the resuscitation fluid allows for dilution of the potassium ion concentration in stored transfused blood. As a result, high concentrations of potassium ion and potential cardiac arrhythmias and cardiac insufficiency caused thereby can be more easily controlled. The resuscitation fluid containing a subphysiological amount of potassium is also useful for purposes of blood substitution and low temperature maintenance of a subject.

In one embodiment, the concentration of chloride ion is in the range of 70 mM to 160 mM. In another embodiment, the concentration of chloride ion is in the range of 110 mM to 125 mM.

Other sources of ions include sodium salts (e.g., sodium hydrogen carbonate, sodium citrate, sodium lactate, sodium sulfate, sodium dihydrogen phosphate, disodium hydrogen phosphate, sodium acetate, sodium glycerophosphate, sodium carbonate, an amino acid sodium salt, sodium propionate, sodium β-hydroxybutyrate, and sodium gluconate), potassium salts (e.g., potassium acetate, potassium gluconate, potassium hydrogen carbonate, potassium glycerophosphate, potassium sulfate, potassium lactate, potassium iodide, potassium dihydrogen phosphate, dipotassium hydrogen phosphate, potassium citrate, an amino acid potassium salt, potassium propionate, and potassium β-hydroxybutyrate), calcium salts (e.g., calcium gluconate, calcium lactate, calcium glycerophosphate, calcium pantothenate, and calcium acetate), magnesium salts (e.g., magnesium sulfate, magnesium glycerophosphate, magnesium acetate, magnesium lactate, and an amino acid magnesium salt), ammonium salts, zinc salts (e.g., zinc sulfate, zinc chloride, zinc gluconate, zinc lactate, and zinc acetate), iron salts (e.g., iron sulfate, iron chloride, and iron gluconate), copper salts (e.g., copper sulfate), and manganese salts (for example, manganese sulfate). Among those, particularly preferable are sodium chloride, potassium chloride, magnesium chloride, disodium hydrogen phosphate, dipotassium hydrogen phosphate, potassium dihydrogen phosphate, sodium lactate, sodium acetate, sodium citrate, potassium acetate, potassium glycerophosphate, calcium gluconate, calcium chloride, magnesium sulfate, and zinc sulfate.

### Nutritive Substances (Carbohydrates and Amino Acids)

The resuscitation fluid may also contain a carbohydrate or a mixture of carbohydrates. Suitable carbohydrates include, but are not limited to, simple hexose (e.g., glucose, fructose and galactose), mannitol, sorbitol or others known to the art. In one embodiment, the resuscitation fluid includes physiological levels of a hexose. “Physiological levels of a hexose” includes a hexose concentration of between 2 mM to 50 mM. In one embodiment, the resuscitation fluid contains 5 mM glucose. At times, it is desirable to increase the concentration of hexose in order to provide nutrition to cells. Thus the range of hexose may be expanded up to about 50 mM if necessary to provide minimal calories for nutrition.

Other suitable carbohydrates include various saccharides to be used for medicinal purposes. Examples of the saccharides include xylitol, dextrin, glycerin, sucrose, trehalose, glycerol, maltose, lactose, and erythritol.

Amino acids known to prevent apiptosis and to provide nutrition also may be included. Examples of such amino acids include glutamine, glycine, proline and 2-aminopentaenoic acid.

### Buffering Agent

The resuscitation fluid of the present invention may further comprise a biological buffer to maintain the pH of the fluid at the physiological range of pH 7-8. Examples of biological buffers include, but are not limited to, N-2-Hydroxyethylpiperazine-N′-2-hydroxypropanesulfonic acid (HEPES), 3-(N-Morpholino)propanesulfonic acid (MOPS), 2-([2-Hydroxy-1,1-bis(hydroxymethyl)ethyl]amino)glyci ethanesulfonic acid (TES), 3-[N-tris(Hydroxy-methyl)methylamino]-2-hydroxyethyl]-1-piperazinep ropanesulfonic acid (EPPS), Tris [hydrolymethyl]-aminoethane (THAM), and Tris [Hydroxylmethyl]methyl aminomethane (TRIS).

In one embodiment, the buffering agent is histidine, imidazole, substituted histidine or imidazole compounds retaining the amphoteric site of the imidazole ring, oligopeptides containing histidine, or mixtures thereof. Histidine is also capable of reducing reactive oxygen species (see e.g., Simpkins et al., J. Trauma. 2007, 63:565-572). Histidine or imidazole may be used in a concentration range of about 0.0001M to about 0.2M, preferably about 0.0001M to about 0.01M.

In another embodiment, the resuscitation fluid of the present invention uses normal biological components to maintain in vivo biological pH. Briefly, some biological compounds, such as lactate, are capable of being metabolized in vivo and act with other biological components to maintain a biologically appropriate pH in an animal. The biological components are effective in maintaining a biologically appropriate pH even at hypothermic temperatures and at essentially bloodless conditions. Examples of the normal biological components include, but are not limited to carboxylic acids, salt and ester thereof. Carboxylic acids have the general structural formula of RCOOX, where R is an alkyl, alkenyl, or aryl, branched or straight chained, containing 1 to 30 carbons which carbons may be substituted, and X is hydrogen or sodium or other biologically compatible ion substituent which can attach at the oxygen position, or is a short straight or branched chain alkyl containing 1-4 carbons, e.g., —CH₃, —CH₂ CH₃. Examples of carboxylic acids and carboxylic acid salts include, but are not limited to, lactate and sodium lactate, citrate and sodium citrate, gluconate and sodium gluconate, pyruvate and sodium pyruvate, succinate and sodium succinate, and acetate and sodium acetate.

### Coagulation Enhancers

Aggressive high volume resuscitation, without controlling the bleeding, can exacerbate the hemorrhage by disrupting the early formed soft thrombi, and by diluting coagulation factors. In certain embodiments, the resuscitation fluid may further comprise one or more coagulation enhancers. Examples of coagulation factors include, but are not limited to, factor 7, thrombin and platelets. These factors may be from natural or non-natural sources. In certain embodiments, factor 7 is added to the resuscitation fluid at a concentration of 70-150 IU/kg, prothrombin complex is added to the resuscitation fluid at a concentration of 15-40 IU/kg, and fibrinogen is added to the resuscitation fluid at a concentration of 50-90 mg/kg.

### Antioxidants

In certain embodiments, the resuscitation fluid may further comprise one or more antioxidants. Examples of antioxidants include, but are not limited to, sodium hydrogen sulfite, sodium sulfite, sodium pyrosulfite (e.g., sodium metabisulfite), rongalite (CH2OHSO2Na), ascorbic acid, sodium ascorbate, erythorbic acid, sodium erythorbate, cysteine, cysteine hydrochloride, homocysteine, glutathione, thioglycerol, α-thioglycerin, sodium edetate, citric acid, isopropyl citrate, potassium dichloroisocyanurate, sodium thioglycolate, sodium pyrosulfite 1,3-butylene glycol, disodium calcium ethylenediaminetetraacetate, disodium ethylenediaminetetraacetate, an amino acid sulfite (e.g, L-lysine sulfite), butylhydroxyanisole (BHA), butylhydroxytoluene (BHT), propyl gallate, ascorbyl palmitate, vitamin E and derivatives thereof (e.g., dl-α-tocopherol, tocopherol acetate, natural vitamin E, d-δ-tocopherol, mixed tocopherol, and trolox), guaiac, nordihydroguaiaretic acid (NDGA), L-ascorbate stearate esters, soybean lecithin, palmitic acid ascorbic acid, benzotriazol, and pentaerythrityl-tetrakis[3-(3,5-di-t-butyl-4-hydroxyphenyl)propionate]2-mercaptobenzimidazole. Among those, preferable are sodium hydrogen sulfite, sodium sulfite, ascorbic acid, homocysteine, dl-α-tocopherol, tocopherol acetate, glutathione, and trolox.

### Other Components

In addition to the components discussed above, the resuscitation fluid may further comprise other additives that include, but are not limited to, antibiotics, such as penicillin, cloxacillin, dicloxacillin, cephalosporin, erythromycin, amoxicillin-clavulanate, ampicillin, tetracycline, trimethoprim-sulfamethoxazole, chloramphenicol, ciprofloxacin, aminoglycoside (e.g., tobramycin and gentamicin), streptomycin, sulfa drugs, kanamycin, neomycin, land monobactams; anti-viral agents, such as amantadine hydrochloride, rimantadin, acyclovir, famciclovir, foscarnet, ganciclovir sodium, idoxuridine, ribavirin, sorivudine, trifluridine, valacyclovir, vangancyclovir, pencyclovir, vidarabin, didanosine, stavudine, zalcitabine, zidovudine, interferon alpha, and edoxudine; anti-fungal agents such as terbinafine hydrochloride, nystatin, amphotericin B, griseofulvin, ketoconazole, miconazole nitrate, flucytosine, fluconazole, itraconazole, clotrimazole, benzoic acid, salicylic acid, voriconazole, caspofungin, and selenium sulfide; vitamins, amino acids, vessel expanders such as alcohols and polyalcohols, surfactants, antibodies against harmful cytokines such as tumor necrosis factor (TNF) or interleukins, and mediators of vascular potency, such as prostaglandins, leukotrienes, and platelet activating factors.

In certain embodiments, the resuscitation fluid further contains, in addition to oxygen, an effective amount of one or more lipophilic gases (i.e., non-oxygen gases having a higher solubility in a hydrophobic medium, such as oil, than in a hydrophilic medium, such as water) that are important in the regulation of vascular function and cellular metabolism. Examples of such gases include nitric oxide, carbon monoxide, hydrogen sulfide and Xenon. Nitirc oxide has been shown to play a significant role in the maintaining the patency of the microcirculation. Nitirc oxide can be very helpful for opening the microcirculation in patients with shock, sickle cell anemia, peripheral vascular disease and stroke. Carbon monoxide has been shown to have an anti-apoptotic effect and cytoprotective properties. Carbon monoxide can be used to prevent the development of pathologic conditions such as ischemia reperfusion injury. Hydrogen sulfide is a regulator of blood pressure. Xenon has been used as a general anesthetic and has been found to have neuroprotective effect. Xenon can be used to ameliorate brain injury or stroke.

In one embodiment, the resuscitation fluid contains micelles loaded with a gas mixture (e.g., a mixture of oxygen, carbon monoxide and/or nitric oxide). In another embodiment, the resuscitation fluid contains a mixture of micelles loaded with various gases. For example, the mixture of micelles may contain 50% NO-loaded micelles and 50% O₂-loaded micelles.

In certain embodiments, the resuscitation fluid may further contain beneficial anions such as lactate or glutamate. Hypertonic lactate containing compositions have been found to be effective in reducing brain edema in patients with acute hemodynamic distress. In one embodiment, the resuscitation fluid contains 250 to 2400 mM of lactic acid or lactate. In another embodiment, the resuscitation fluid contains 250 to 2400 mM of lactic acid or lactate and 2 to 10 mM potassium.

In certain embodiments, the resuscitation fluid further contain anti-cancer drugs and/or intracellular signal molecules, such as cAMP and diacylglycerol. In other embodiments, the resuscitation fluid further contain one or more organelles or organelle components such as endoplasmic reticulum, ribosomes, and mitochondria in whole or in part.

The resuscitation fluid possesses the ability to absorb toxic chemical molecules/biomolecules produced as the result of trauma or hemorrhagic shock. For example, lymph factors produced in gut and thoracic duct lymph nodes may result in acute lung injury and red blood cell deformability after trauma/hemorrhagic shock. Other toxic chemical molecules/biomolecules include, but are not limited to, leukotrienes, prostaglandins, nitric oxide, endotoxin and tumor necrosis factor (TNF). The lipid emulsion in the resuscitation fluid allows effective absorption of lipophilic chemical molecules/biomolecules. In certain embodiment, the resuscitation fluid further contains antagonists to toxic chemical molecules/biomolecules, such as antibodies to endotoxins.

### Preparation of the Resuscitation Fluid

The resuscitation fluid may be prepared by mixing the lipid component, the aqueous carrier, and any other components to form an emulsion. Commonly used mixing methods include, but are not limited to, stirring, shaking, homogenization, vibration and sonication. In one embodiment, the resuscitation fluid is formed by mixing a pre-formed lipid emulsion, such as Infralipid®, with the aqueous carrier and other components. In addition, the oxygen carrying lipid can be carried in a liposome, glycosylated liposomes, or erythrocyte ghosts.

In order to increase the oxygen content in the resuscitation fluid, the resuscitation fluid may be oxygenated by bubbling pure oxygen or a gas with an oxygen content in the range of 21% to 100% (v/v), 40% to 100% (v/v), 60% to 100% (v/v), 80% to 100% (v/v) or 90% to 100% (v/v) through the resuscitation fluid for a period of 30 seconds or longer, preferably 1-15 minutes, more preferably 1-5 minutes. The oxygenation time for a resuscitation fluid of a particular composition may be determined experimentally. In one embodiment, the resuscitation fluid is oxygenated immediately prior to application.

In one embodiment, the resuscitation fluid comprises an oxygenated lipid emulsion. As used herein, the term “oxygenated lipid emulsion’ or “oxygenated resuscitation fluid” refers to a specific type of gassed lipid emulsion or gassed resuscitation fluid which has been forced to absorb oxygen such that the total concentration of oxygen contained therein is greater than that present in the same liquid at atmospheric equilibrium conditions.

### Kits

Another aspect of the present invention relates to a resuscitation kit. In one embodiment, the resuscitation kit comprises an oxygenated resuscitation fluid and at least one additive. Examples of additives include, but are not limited to, oncotic agent, crystalloid agent, vessel expander, cardioplegic, or cardiotonic agent scavengers of free radicals or mediators, cell signaling modulators, and receptor agonists or antagonists. In another experiment, the kit further contains an intravenous infusion (IV) set. In another embodiment, the oxygenated resuscitation fluid is contained in one or more preloaded syringes for emergency application. In another embodiment, the kit further contains an oxygen container that can be used to re-oxygenate the resuscitation fluid immediately prior to application. The oxygen container may contain pure oxygen, or a gas mixture of oxygen with one or more other lipophilic gases such as hydrogen sulfide, carbon monoxide, nitric oxide and Xenon. In another embodiment, the kit contains a resuscitation fluid, and an air pump for oxygenating the resuscitation fluid with ambient air immediately prior to application.

In another embodiment, the kit contains an oxygen producing canister that contains, a material that is capable of producing oxygen through a chemical reaction. Materials that may be used for the production of oxygen include, but are not limited to, sodium chlorate, sodium peroxide and potassium superoxide.

### Treatment Methods

Another aspect of the present invention relates to a method for treating conditions related to lack of blood supply with a lipid-based resuscitation fluid. Conditions related to a lack of blood supply include, but are not limited to, hypovolemia, ischemia, hemodilution, trauma, septic shock, cancer, anemia, cardioplegia, hypoxia and organ perfusion. The term “hypovolemia,” as used herein, refers to an abnormally decreased volume of circulating fluid (blood or plasma) in the body. This condition may result from “hemorrhage,” or the escape of blood from the vessels. The term “ischemia,” as used herein, refers to a deficiency of blood in a part of the body, usually caused by a functional constriction or actual obstruction of a blood vessel.

The resuscitation fluid may be administered intravenously or intraarterially to a subject in need of such treatment. Administration of the resuscitation fluid can occur for a period of seconds to hours depending on the purpose of the resuscitation fluid usage. For example, when used as a blood volume expander and an oxygen carrier for the treatment of severe hemorrhage shock, the usual time course of administration is as rapidly as possible, which may range from about 1 ml/kg/hour to about 15 ml/kg/min.

While the resuscitation fluid of the present invention is being administered to and circulated through the subject, various agents such as cardioplegic or cardiotonic agents may be administered either directly into the subject's circulatory system, administered directly to the subject's myocardium, or added to the resuscitation fluid of the present invention. These components are added to achieve desired physiological effects such as maintaining regular cardiac contractile activity, stopping cardiac fibrillation or completely inhibiting contractile activity of the myocardium or heart muscle.

Cardioplegic agents are materials that cause myocardial contraction to cease and include anesthetics such as lidocaine, procaine and novocaine and monovalent cations such as potassium ion in concentrations sufficient to achieve myocardial contractile inhibition. Concentrations of potassium ion sufficient to achieve this effect are generally in excess of 15 mM.

During revival of a subject, the subject may be re-infused with a mixture of the resuscitation fluid described along with blood retained from the subject or obtained from blood donors. Whole blood is infused until the subject achieves an acceptable hematocrit, generally exceeding hematocrits of about 30%. When an acceptable hematocrit is achieved, perfusion is discontinued and the subject is revived after closure of surgical wounds using conventional procedures. In certain embodiments, the resuscitation fluid of the present invention is used to treat all forms of shocks, including but are not limited to, neurogenic shock, cardiogenic shock, adrenal insufficiency shock and septic shock.

Another aspect of the present invention relates to a method of using the resuscitation fluid described above to oxygenate patients whose lungs are severely damaged and unable to absorb oxygen even with special modes of ventilation. The oxygen loaded resuscitation fluid may deliver oxygen to tissues via circulation and allows the lung to recover from the damage. In this regard, the resuscitation fluid may be used to replace extracorporeal membrane oxygenation (ECMO).

Another aspect of the present invention relates to a method of using the resuscitation fluid in exchange transfusion and whole circulation perfusion to wash out blood containing harmful materials such as infectious agents, cancerous agents, and toxic agents. In both cases, the resuscitation fluid may comprise an emulsion consisting of 20% lipid micelles and isotonic saline with or without albumin. The resuscitation fluid may also be used to absorb toxic chemical molecules/biomolecules produced as the result of trauma, hemorrhagic shock or other form of shocks. After perfusion with the resuscitation fluid, whole blood is infused until an acceptable hematocrit is achieved.

The resuscitation fluid may be loaded with oxygen or another gas such as NO, CO or Xe as needed. The resuscitation fluid may further contain one or more additives such as clotting enhancing factors, anti-infection agents, intracellular signal molecules such as cAMP or diacylglycerol, and anti-cancer drugs. In certain embodiments, the resuscitation fluid further contain one or more organelles or organelle components such as endoplasmic reticulum, ribosomes, and mitochondria in whole or in part.

Another aspect of the present invention relates to a method of preserving the biological integrity of organs of a mammalian donor organism. using the resuscitation fluid described. In one embodiment, the subject organ is chilled and the resuscitation fluid is perfused into the subject organ using a pumped circulating device such as a centrifugal pump, roller pump, peristaltic pump or other known and available circulatory pump. The circulating device is connected to the subject organ via cannulae inserted surgically into appropriate veins and arteries. When the resuscitation fluid is administered to a chilled subject organ, it is generally administered via an arterial cannula and removed from the subject via a venous cannula and discarded or stored.

When used for organ perfusion during an organ transplantation, the resuscitation fluid may be administered slowly over a period of hours.

### EXAMPLES

The following example is put forth so as to provide those of ordinary skill in the art with a complete disclosure and description of how to carry out the method of the present invention and is not intended to limit the scope of the invention. Efforts have been made to ensure accuracy with respect to numbers used (e.g., amounts, temperature, etc.), but some experimental error and deviation should be accounted for. Unless indicated otherwise, parts are parts by weight, molecular weight is weight average molecular weight, temperature is in degrees Centigrade, and pressure is at or near atmospheric.

### Example 1

### Methods and Materials

Lipid emulsion: 20% Intralipid (marketed and sold by Baxter International Inc., Deerfield, Ill.) was used as a model lipid emulsion. It is composed of 20% soy bean oil, 1.2% egg yolk phospholipids 2.25% glycerin, water and sodium hydroxide to adjust the pH to 8.

Determination of oxygen content of Intralipid: Samples of distilled water, Ringer's lactate (RL) and Intralipid (20%) (1 ml each) were left open to air in 2.0 ml tubes for 30 minutes prior to dissolved gas analysis. Volumes of 50 uL drawn from each of these fluids were injected into a Sievers purge vessel at 37° C. containing 36 ml of a mildly acidic solution consisting of 32 ml of 1M HCL and 4 ml of 0.5M ascorbic acid. The solution was continuously purged with high purity helium to transport any oxygen released from the samples to a mass spectrometer (HP 5975) for direct gas analysis. Signals generated at m/z=32 upon injection of RL and lipid emulsion samples were integrated using Peakfit and compared to those obtained with distilled water.

Animals and animal procedures: Male and female mice weighing 27-47 grams were utilized. The strains were either CD-1 or NFR2. All comparisons utilized the same strain. Mice were anesthetized using ketamine/xylazine anesthesia administered subcutaneously. In order to prevent the skewing of data due to the cardiodepressant effects of the anesthetic agent, the experiment was aborted and the mouse euthanized in the rare instance when more anesthetic was required than the calculated dose. Once it was clear that the mouse was well-anesthetized, the carotid artery was cannulated. As much blood as possible was removed in one minute. This resulted in the loss of 55% of blood volume and 100% lethality without any infusion. Immediately after blood removal infusions were administered over one minute.

Either RL or Intralipid was administered at a volume equal to the amount of blood that had been removed. Blood pressure was measured at the carotid artery using a BP-2 monitor made by Columbus Instruments (Columbus, Ohio). This monitor measures the blood pressure as a voltage. A standard curve was prepared. Measured voltages were converted to blood pressure (BP) using the following formula:

BP=[Voltage−0.1006]/0.0107

No warming measures were applied to the mice. No measures were taken to support respiration.

Statistical Analysis Data were analyzed using Student's unpaired t test.

### Example 2

### Oxygen Content of the Resuscitation Fluid

Intralipid® 20% I.V. Fat Emulsion (marketed and sold by Baxter International Inc., Deerfield, Ill.) was used as a sample resuscitation fluid (RF). The composition of Intralipid® is 20% soybean oil, 1.2% egg yolk phospholipids, 2.25% glycerin, water and sodium hydroxide to adjust the pH to 8. Oxygen content in the RF was measured using mass spectrometry. As shown in Table I, the oxygen content of the RF was nearly twice that of Ringer's lactate (RL), a standard resuscitation fluid infused when a large amount of blood is lost. The oxygen content of RL was equivalent to that of water. As shown in Table II, the oxygen content of the RF was increased five-fold by bubbling oxygen through it for approximately 1 minute. After oxygen loading, the oxygen content of RF compared favorably to that of blood with the minimum acceptable hemoglobin level (i.e., 7.0 g/dl). Table III shows that theoretical oxygen content in RF with higher lipid contents.

TABLE I
Oxygen content of Ringer's lactate and Intralipid ® 20%

| | Ringer's lactate | Intralipid ® 20% |
|---|---|---|
| Oxygen Content* | 0.91 ± 0.11* | 1.78 ± 0.09* |

*the oxygen content is expressed as the amount relative to the oxygen content in water.

TABLE II
Oxygen solubility in various liquids at 1 atm
Oxygen Content at 25° C. and Sea level Pressure

| | |
|---|---|
| Blood (hemoglobin of 7.0) | 72.8 mg/L |
| Water | 8.3 mg/L |
| LM (20%) | 15.1 mg/L |
| LM (20% after oxygen perfusion) | 75.5 mg/L |

TABLE III
Theoretical oxygen content in RF with higher lipid concentrations
Theoretical oxygen content at higher concentrations

| | |
|---|---|
| LM (40%) | 24.9 mg/L |
| LM (40% after oxygen perfusion) | 124.5 mg/L |
| LM (60%) | 33.2 mg/L |
| LM (60% after oxygen perfusion) | 166.0 mg/L |

### Example 3

### The Effect of Resuscitation Fluid in Restoring Arterial Pressure in Mice with Severe Hemorrhagic Shock

The effect of the RF in Example 2 on blood pressure was determined in mice. Mice were anesthetized and a cannula was placed into the carotid artery. All the blood that could be removed was removed via the carotid artery. After the blood was removed a volume of either RL or RF was given equal to the amount of blood removed. 6 mice were in the RF group and 6 mice were in the RL group. The observation period was one hour. Two of the mice given RL died within ten minutes. All mice given RF lived through the entire hour observation period and until euthanized at 1-4 hours. Animals were euthanized whenever they began to awaken from the anesthesia or at the end of the observation period to prevent suffering.

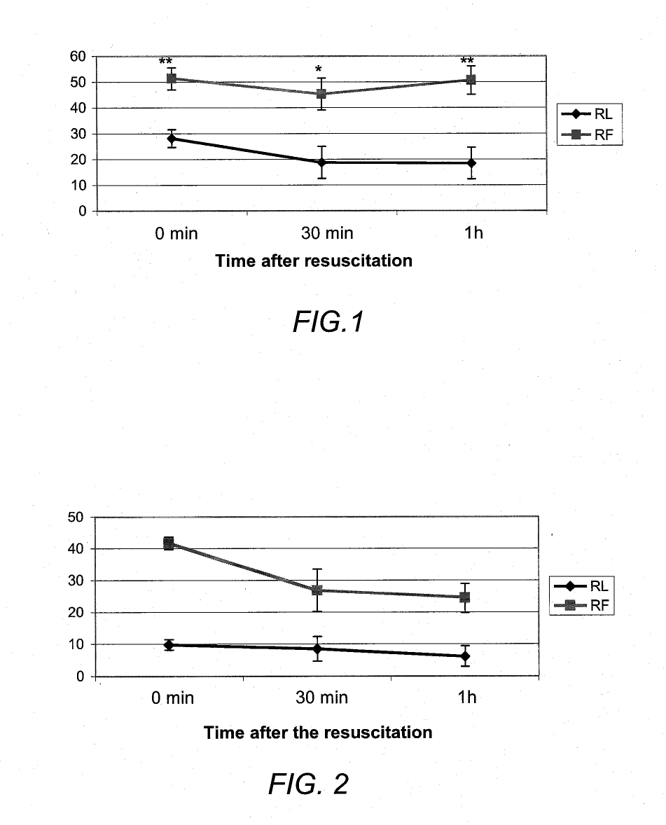

FIGS. 1 and 2 show the difference between the systolic blood pressure (FIG. 1) and diastolic blood pressure (FIG. 2) after hemorrhage and after infusion of RL or RF at time=0, 30 and 60 minutes. The Y axis represents the blood pressure attained after infusion minus the blood pressure after hemorrhage in mm of Hg. The X axis shows the specific time after the infusion. All data were analyzed for statistical significance using an unpaired two tailed t test. These graphs show that RF raised the blood pressure higher than RL.

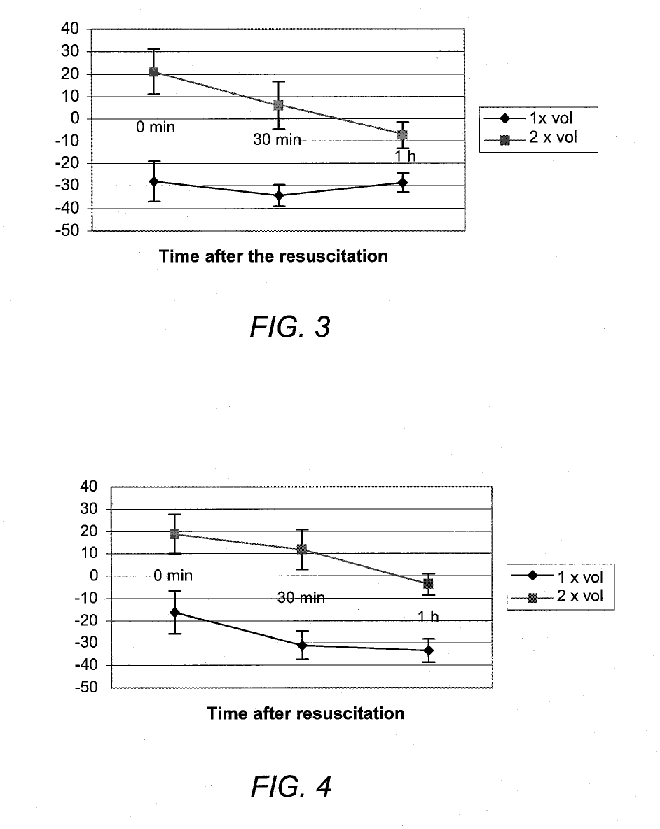

In another experiment, RF at a volume twice the amount of blood removed was given. This led to an even greater increase in the blood pressure as shown in FIGS. 3 and 4. The points on the graph represent the mean of 6 mice+/−SE. The Y-axis shows the difference between the systolic blood pressure (FIG. 3) and diastolic blood pressure (FIG. 4) after infusion of RF at 1× the blood volume (diamond) or 2× the blood volume (square) minus the baseline pressure prior to hemorrhage in mm of Hg. Under this scheme therefore, 0 represents the blood pressure at the beginning of the experiment before hemorrhage. The X axis shows specific times after the infusion. 2× the blood volume raised the blood pressure higher than the pressure reached after infusion of 1× the blood volume (p<0.01). Moreover, the pressure achieved after infusion of 2× the removed blood volume exceeded the pressure that existed prior to hemorrhage.

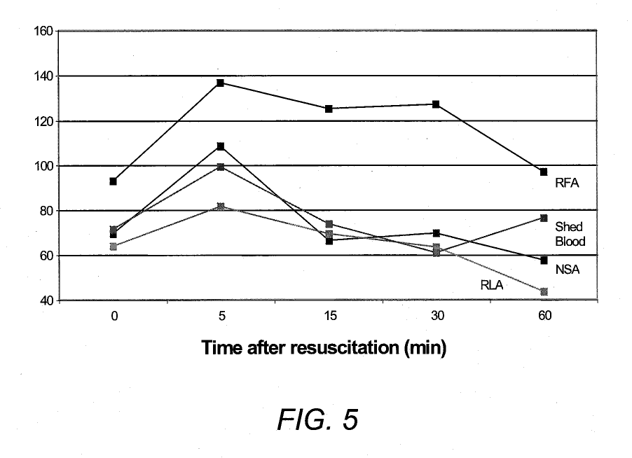

In another experiment, a resuscitation fluid containing Intralipid® 20% and 5% (w/v) albumin was prepared by dissolving albumin (Sigma Aldrich, 99% pure, fatty acid free, essentially globulin free, catalog number A3782-5G) in Intralipid® 20% to a final concentration of 50 mg/ml. The new resuscitation fluid with albumin (RFA) was tested using the experimental procedure described above. Albumin dissolved in normal saline (NSA) and Ringer's lactate (RLA) at 50 mg/ml, as well as the shed blood (i.e., the blood that had been removed from the mice), were used as controls. In FIG. 5, the Y axis shows the systolic blood pressure (expressed as percentage of mean pre-hemorrhage blood pressure) achieved by infusion of the various fluids. The X axis shows specific times after the infusion. The data show that RFA is superior even to shed blood in maintaining blood pressure. Similar results were also obtained for the diastolic blood pressure (not shown). For each time point, an average of 6-7 mice is plotted. Differences between shed blood and RFA was statistically significant (P<0.05) at 5, 15 and 30 minutes.

These experimental results are consistent with the fact that the lipid micelles in the resuscitation fluid are capable of exerting an osmotic force and absorbing mediators of vascular potency, such as prostaglandins, nitric oxide, leukotrienes, and platelet activating factors.

The terms and descriptions used herein are set forth by way of illustration only and are not meant as limitations. Those skilled in the art will recognize that many variations are possible within the spirit and scope of the invention as defined in the following claims, and their equivalents, in which all terms are to be understood in their broadest possible sense unless otherwise indicated.

What is claimed is:

1. A method for treating conditions related to lack of blood supply in a human or animal subject, comprising:
administering to said subject an effective amount of a resuscitation fluid comprising a lipophilic component and a polar liquid carrier, wherein said lipophilic component forms an emulsion with said polar liquid carrier.

2. The method of claim 1, wherein said conditions related to the lack of blood supply comprise hypovolemia and ischemia.

3. The method of claim 1, wherein said resuscitation fluid further comprises approximately 5% (w/v) albumin.

4. The method of claim 1, wherein said lipid component forms micelles in the emulsion.

5. The method of claim 1, wherein said lipid component forms liposomes in the emulsion.

6. The method of claim 1, wherein said resuscitation fluid is an oxygenated resuscitation fluid.

7. The method of claim 1, further comprising:
oxygenating the resuscitation fluid prior to administration to said subject.

8. The method of claim 7, wherein the step of oxygenating the resuscitation fluid comprising bubbling an oxygen-containing gas through the resuscitation fluid for 1 to 5 minutes.

9. The method of claim 8, wherein said oxygen-containing gas comprises 80%-100% (v/v) oxygen.

10. The method of claim 1, wherein said resuscitation fluid comprises an effective amount of oxygen and a lipophilic gas for regulation of vascular function and cellular metabolism, said lipophilic gas is selected from the group consisting of hydrogen sulfide, nitric oxide, carbon monoxide and xenon.

11. A method for treating trauma or shock in a human or animal subject, comprising:
administering to said subject an effective amount of a resuscitation fluid comprising an oxygenated lipophilic component.

12. The method of claim 11, wherein said oxygenated lipophilic component is encapsulated in micelles.

13. The method of claim 11, wherein said oxygenated lipophilic component is encapsulated in liposomes.

14. The method of claim 11, wherein said oxygenated lipophilic component is encapsulated in erythrocyte ghost.

15. The method of claim 11, wherein said oxygenated lipophilic component further comprises an effective amount of a gas for regulation of vascular function and cellular metabolism, said gas is selected from the group consisting of hydrogen sulfide, nitric oxide and carbon monoxide.

16. A method for removing a lipophilic harmful material from the blood circulation of a human or animal, comprising:
perfusing said human or animal with an effective amount of a resuscitation fluid comprising a lipophilic component and a polar liquid carrier; and
infusing said human or animal with whole blood until an acceptable hematocrit is achieved.

17. A method for preserving the biological integrity of an organ of a mammalian donor organism, comprising:
perfusing said organ with an effective amount of a resuscitation fluid comprising an oxygenated lipophilic component and a polar liquid carrier.

18. A resuscitation fluid, comprising:
an oxygenated lipophilic component carried by micelles, liposomes and/or erythocyte ghost; and
a buffering agent.

19. The resuscitation fluid of claim 18, further comprising a plasma component.

20. The resuscitation fluid of claim 19, wherein said lipid component comprises 20% (w/v) purified soybean oil, 1.2% (w/v) purified egg phospholipids, and 22% (w/v) glycerol anhydrous, and wherein said plasma component is approximately 5% (w/v) albumin.

21. The resuscitation fluid of claim 18, wherein the buffering agent comprises histidine.

22. The resuscitation fluid of claim 18, further comprising an effective amount of a lipophilic gas other than oxygen.

23. The resuscitation fluid of claim 22, wherein said lipophilic gas is selected from the group consisting of hydrogen sulfide, carbon monoxide, nitric oxide and xenon.

24. The resuscitation fluid of claim 18, wherein said resuscitation fluid is free of hemoglobin.

25. A resuscitation kit, comprising:
a resuscitation fluid comprising a lipophilic component and a polar liquid carrier, and
an oxygenation device.

26. The resuscitation kit of claim 25, wherein said oxygenation device is a container containing an oxygenating gas.

27. The resuscitation kit of claim 26, wherein said oxygenating gas comprises oxygen and one or more gases selected from the group consisting of hydrogen sulfide, carbon monoxide, nitric oxide and xenon.

28. The resuscitation kit of claim 25, wherein said oxygenation device contains a material that is capable of generating oxygen through a chemical reaction.

29. The resuscitation kit of claim 25, wherein said oxygenation device comprises an air pump.

30. The resuscitation kit of claim 25, further comprising an intravenous infusion (IV) set.
